(19) United States
(12) Patent Application Publication
(10) Pub. No.: US 2014/0214014 A1
(43) Pub. Date: Jul. 31, 2014

(54) LASER DEPILATORY DEVICE

(71) Applicant: YA-MAN LTD, Tokyo (JP)

(72) Inventor: Iwao YAMAZAKI, Tokyo (JP)

(21) Appl. No.: 14/228,563

(22) Filed: Mar. 28, 2014

Related U.S. Application Data
(63) Continuation of application No. PCT/JP2012/006118, filed on Sep. 26, 2012.

(30) Foreign Application Priority Data
Oct. 3, 2011 (JP) 2011-219378

(51) Int. Cl. A61B 18/20

(52) U.S. Cl. CPC A61B 18/203
USPC 606/9

(57) ABSTRACT

A laser depilatory device is provided where the tips of skin hairs can be trimmed evenly after cauterization and the skin hair can he removed efficiently with lower power consumption, while preventing the direct adverse influent of het on the human skim The laser device includes: a housing including a contacting surface to which a skin surface is contacted and a recessed portion formed on the contacting surface; a laser light generating section disposed in the housing for generating laser light; and a first hole portion formed on an inner wall surface of the recessed portion for emitting the laser light through a space inside the recessed portion towards the inner wall.

### CROSS-REFERENCE TO RELATED APPLICATIONS

This application is a continuation of prior International Application No. PCT/JP2012/006118 filed on Sep. 26, 2012, which is based upon and claims the benefit of priority from Japanese Patent Application No. 2011-219378 filed on Oct. 3, 2011; the entire contents of all of which are incorporated herein by reference.

### FIELD

Embodiments described herein relate generally to a laser depilatory device for removing skin hair with irradiating laser light.

### BACKGROUND

Conventionally, a non-electric powered device such as a razor or a tweezer has been known as a device which removes hair such as facial hair or leg and arm hair (i.e., hair removal or hair depilation). The usage of this kind of non-electric powered device frequently entails botheration and a discomfort feeling. The usage of this kind of non-electric powered device also imparts a direct stimulation or a damage to a skin. On the other hand, a so-called dry shaver is known to be provided as an electromechanical device. This kind of dry shaver is capable of being used inside a vehicle. In this kind of dry shaver, an electromechanical razor is widely used which does not impart a significant damage to a skin. In the electromechanical razor, a rotational blade is configured to be rotated by, e.g., a motor. For this reason, the electromechanical razor sometimes generates a noise and therefore adversely affects a surrounding environment.

Additionally, it has been proposed a hair removal device of so-called a heat generator which cauterizes a skin hair grown on a skin by use of a wire or a ribbon heated at high temperature adjacent to the skin. The heat generator is capable of removing skin hair without generating a noise. The heat generator provides a heat pulse with respect to the wire or the ribbon which serves as a heating element. Thus, an average temperature can be lowered even if the temperature has been once raised at a moment. As a result, the heat generator has an advantage that a damage due to a heat is not imparted to the skin.

### SUMMARY

However, in the above mentioned conventional hair removal device, there is a case where it takes a lone: time to remove skin hair by cauterization because the heating element is a wire or a ribbon for applying heat for skin hair removal. For example, the wire or the ribbon has a heat resistance so that it sometimes takes a long time for a temperature of the wire or the ribbon to reach a temperature at which the skin hair can be cauterized. Additionally, in the above mentioned conventional hair removal device, a speed for cauterization varies depending on a thickness of hair so that tips of hairs after cauterization may be unevenly aligned. Furthermore, in case the wire or the ribbon is employed, a disadvantage may be caused that a power consumption is increased.

The embodiment is made in light of the above mentioned circumstances, and an object of the embodiment is therefore to provide a laser depilatory device that can utilize a rectilinear and convergent characteristics of laser light to evenly align tips of hairs after cauterization without adversely affecting a human skin due to a direct heat application. Thus, the embodiment can achieve a hair removal in an efficient manner with saving power consumption.

In order to achieve the above object, according to an aspect of the embodiment, there is provided a laser depilatory device comprising: a housing including a contacting surface to which a skin surface is contacted and a recessed portion formed on the contacting surface; a laser light generating section disposed in the housing for generating laser light; and a first hole portion formed on an inner wall surface of the recessed portion for emitting the laser light through a space inside the recessed portion towards the inner wall.

According to another aspect of the embodiment, the laser depilatory device may include a second hole portion formed on an inner wall surface of the recessed portion for emitting the laser light through a space inside the recessed portion towards the inner wall in a direction different from a direction of the laser light emitted from the first hole portion.

According to yet another aspect of the embodiment, the laser depilatory device may include a tubular laser light deriving section connected to the laser light generating section and the first hole portion for transmitting the laser light generated by the laser light generating section to the first hole portion.

According to yet another aspect of the embodiment, the first hole portion may be arranged in a straight line along the contacting surface. With this configuration, the laser depilatory device according to the embodiment enables skin hair guided inside the recessed portion to be simultaneously and yet efficiently cauterized along the contacting surface.

According to yet another aspect of the embodiment, the laser depilatory device includes a slit opposing the first hole portion. With this configuration, a user can utilize the slit as a guide when a body of the laser depilatory device is moved along the user's skin. As a result, the laser depilatory device can guide skin hair inside the recessed portion. Furthermore, the slit allows the skin hair to be raised or groomed. Accordingly, the skin hair can be cut to be aligned at the prescribed height along the contacting surface.

According to yet another aspect of the embodiment, the laser depilatory device includes a heating element arranged substantially in parallel with the first hole portion, a part of which being arranged in contact with the skin surface, for absorbing the laser light emitted from the first hole portion to generate heat. The heating element converts light energy to heat energy to be heated to high temperature. The heat generation of the heating element allows a pore of the skin to open so that a removal function can be improved. Since the heating element is heated with the laser light, the heating element is free from unnecessary power consumption required for the pore of the skin being opened. Furthermore, the heating element absorbs light, therefore it can be prevented that emitted laser light enters into the user's eye.

According to yet another aspect of the embodiment, the laser depilatory device may include a detecting section for detecting the contacting surface being moved towards a direction of the laser irradiated from the first hole portion with respect to the skin surface; and a controller for turning on/off an irradiation of the laser light based on a detection by the detecting section. With this configuration, the laser light is automatically irradiated only when a user carries forward the laser depilatory device for hair removal on the user's skin. On the other hand, an irradiation of the laser light automatically stops when the user stops the laser depilatory device to move or carries backward the laser depilatory device. As a result, a power loss can be suppressed due to a constant irradiation of the laser light.

According to the embodiment, the tips of skin hairs can be trimmed evenly after cauterization, while preventing the direct adverse influent of heat on the human skin. Furthermore, according to the embodiment, the skin hair can be removed effectively with lower power consumption.

### BRIEF DESCRIPTION OF THE DRAWINGS

FIG. 1 is a perspective view showing a laser depilatory device according to the first embodiment.

FIG. 2 is a partially fractured front elevational view showing an outline of an inner equipment of the laser depilatory device according to the first embodiment.

FIG. 3 is a perspective view showing the modification to the first embodiment.

FIG. 4 is a partially fractured front elevational view showing an outline of an inner equipment of the laser depilatory device according to the second embodiment.

FIG. 5 is a perspective view showing the modification to the laser depilatory device.

FIG. 6 is a perspective view showing another modification to the laser depilatory device.

FIG. 7 is a plan view showing yet another modification to the laser depilatory device.

FIG. 8 is a plan view showing yet another modification to the laser depilatory device.

### DETAILED DESCRIPTION

Hereinafter, preferred embodiments will be described in detail with reference to the accompanying drawings.

### First Embodiment

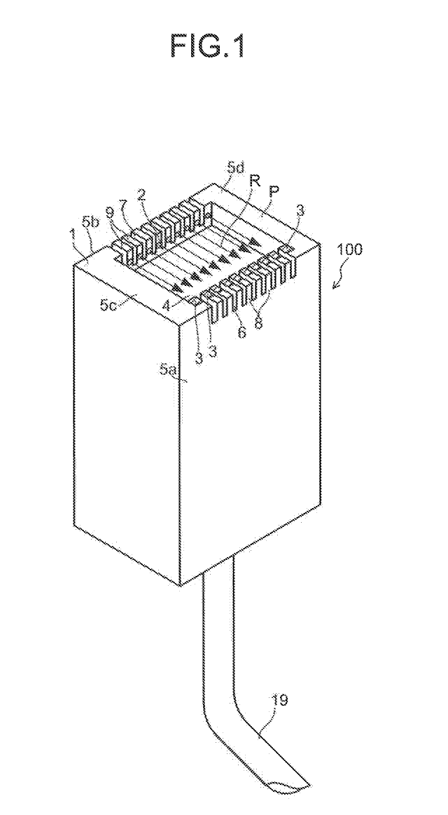

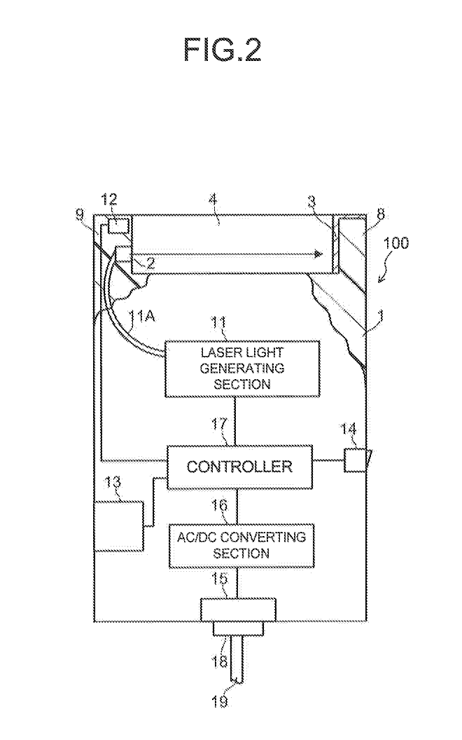

Referring to FIGS. 1 and 2, a laser depilatory device 100 according to the first embodiment comprises a housing 1, a laser light outlet 2 and a heating element 3. The laser depilatory device 100 is capable of cauterizing skin hair with laser light. By use of the laser light, removing skin hair with lower power consumption can be achieved in comparison to the conventional hair removal device that heats a wire or a ribbon.

The housing 1 has cuboid shape which is surrounded by wall sections 5a, 5b, 5c and 5d as a whole. A recessed portion 4 is formed at one end surface of the housing 1 which contacts wall sections 5a, 5b, 5c and 5d, respectively, in an approximately center region of the end surface. The recessed portion 4 is defined by inner walls of the wall sections 5a 5b, 5c and 5d, and has a prescribed depth.

The end surface of the housing 1 (i.e., end portions of the wall sections 5a to 5d), at which the recessed portion 4 is formed, is capable of contacting (i.e., abutting) human skin, such as a skin of a face, or legs and arms, as described hereinafter, and constitutes a smooth surface (i.e., a contacting surface) P.

At rim portions of an opposing pair of wall sections 5a and 5b which constitutes the recessed portion 4, a plurality of slits 6 and 7 are arranged, respectively (nine slits in an illustrative example shown in FIG. 1), at an equal interval along the respective rim portions of the recessed portion 4. The slits 6 and 7 communicate between an inner wall of the recessed portion 4 and an outer wall of the housing 1. The slits 6 and 7 have the same shape and the same dimension with respect to one another. Yet concerning the slits 6 and 7, corresponding slits are positioned at opposing positions each other. As a result, the slits 6 and 7 allow the device to form a plurality of protruding portions 8, 9 each of which has the same shape and the same dimension one another at wall sections 5a and 5b, respectively. The heating element 3 are disposed at each of protruding portions 8 of the wall section 5a, the protruding portions 8 sandwiching the slit 6.

The slits 6 and 7 function as a guide which raises or grooms skin hair subject to the cauterization to guide into the recessed portion 4.

A plurality of laser light outlets 2 are provided, respectively, at an inner side surface of the protruding portions 9 of the wall section 5b which sandwich the slit 7 (i.e., an inner wall surface of the recessed portion 4 opposing the protruding portion 8). The laser light outlets 2, as a whole, are aligned in a straight line along the contacting surface P of the rim portion of the wall section 5b.

The laser light outlet 2 is a bore which is arranged for emitting laser light towards heating elements 3 disposed at protruding portions 8. The laser light emitted from the laser light outlet 2 arranged on the inner side surface of each of the protruding portion 9 (as shown with an arrow R in FIG. 1) is irradiated to the heating element 3 arranged on the inner side surface of each of the protruding portions 8 which corresponds to each of the protruding portions 9. A collective lens may be arranged at the laser light outlet 2. Arranging the collective lens allows the laser light to be effectively irradiated to the heating element 3.

The heating element 3 is arranged such that the heating elements 3 are arranged in series on the inner wall surface of the recessed portion 4 (i.e., the surface opposing to the protruding portion 9) from a part of an upper surface of the protruding portion 8 (i.e., the contacting surface P). The heating elements 3 are mounted such that the heating elements 3, as a whole, have the same width as the protruding portions 8 and the cross sections thereof have inverted L-shape. The heating element 3 is adhered to the upper surface and the inner side surface of the protruding portion 8. The heating element 3 may be integrally disposed with the housing f when the housing 1 is molded.

In case infrared laser is employed as laser light emitted from the laser light outlet 2, the heating element 3 is composed of infrared absorption material, in this case, the heating element 3 is realized by the process that liquid infrared absorption material be applied to the protruding portion 8, or alternatively, sheet-shaped or film-shaped infrared absorption material be pasted on the protruding portion 8.

As shown in FIG. 2, the laser depilatory device 100 according to the first embodiment, in addition to the above described laser light outlets 2 and the heating element 3, a laser light generating section 11, a detecting section 12, a battery 13, a power switch 14, a power receiving connector 15, an AC/DC converting section 16 and a controller 17, all of which are in the housing 1.

The laser light generating section 11 is a device that generates laser light with higher directivity and higher convergence. The laser light generating section 11 is, for example, a pulse laser device with small-diameter, and generates laser light with prescribed wavelength by way of prescribed oscillation method. Solid-state laser, gas laser or semiconductor laser may be employed as a light source of the laser light generating section 11. The output level of the laser light generating section 11 is regulated to a level capable of cauterizing skin hair of human in a small amount of time. The laser light generating section 11 is capable of emitting laser light that can cauterize skin hair upon supply of power. In other words, according to the laser light generating section 11, an amount of time required for cauterizing skin hair can be shortened in comparison to the conventional cauterization with the wire or the ribbon. The laser light generating section 11 is connected to the laser light outlet 2 through light deriving tube 11A which is light guiding elements.

The light deriving tube 11A is a transmission channel of laser light. The light deriving tube 11A derives the laser light generated by the laser light generating section 11 to the laser light outlet 2. The light deriving tube 11A, for example, is made of an optical fiber and has a multi-layered structure. In other words, the laser light generated by the laser light generating section 11 is derived to the laser light outlet 2 with repetitive reflection within the light deriving tube 11A. The laser light derived to the laser light outlet 2 is then irradiated onto the heating elements 3. It should be noted that, although not shown, a light branching filter may be incorporated in the housing 1. The light branching filter branches the laser light venerated by the laser light generating section 11 into a prescribed number of rays of laser light. The branched rays of laser light are emitted from respective laser light outlets 2 through respective light deriving tubes 11A.

The detecting section 12 is a sensor that detects that, electrically, electrostatically or optically, the skin of human be getting adjacent or in contact. The detecting section 12 detects that the contacting surface P of the housing 1 contacts or gets adjacent to the skin of human.

The detecting section 12 is employed for controlling the turn on/off of an irradiation of the laser light onto the heating element 3. For example, user pushes the contacting surface P of the housing 1 against his/her skin so that the detecting section 12 detects a contact with the skin (or an approximation being about to contact the skin). As a result, the laser light generating section 11 generates the laser light. Then, the laser light generated is irradiated onto the heating element 3. When the contacting surface P of the housing 1 is disengaged from the skin, the laser light generating section 11 halts its driving. Consequently, the irradiation of the laser light onto the heating element 3 also halts.

The power switch 14 turns on/off the power supply to the laser light generating unit 11. Alternatively, a function of the power switch 14 may he provided with the detecting section 12. In this case, the power switch 134 may he omitted. In other words, the detecting section 12 detects a contact with or an approximation to the skin of human so that the power supply to the laser light generating section 11 may he turned on/off.

An AC plug 18 to which an AC code 19 is connected is plugged into the power receiving connector 15. When the AC plug 18 is plugged into the power receiving connector 15, the AC/DC converting section 16 converts alternating-current power into direct-current power. Then, the converted direct-current power is supplied to the controller 17.

The controller 17 selects either one of the direct-current power supplied from the AC/DC converting section 16 and the direct-current power output from the battery 13. The controller 17 then supplies the selected direct-current power to the laser light generating section 11.

It should be noted that the controller 17 cutouts the power supply from the battery 13 when the power switch 14 is turned on and the AC plug 18 is plugged into the power receiving connector 15. In other words, the controller 17 supplies to the laser light generating section 11 the direct-current power input through the AC/DC converting section 16. The controller 17 supplies to the laser light generating section 11 the power of the battery 13 when the power switch 14 is turned on and the AC plug 18 is not plugged into the power receiving connector 15.

The controller 17 controls the turn on/off of the driving of the laser light generating section 11 based on a detection signal from the detection section 12. The “detection signal from the detection section 12” is a signal that is output in response to the event that the detection section 12 detects, e.g., the contacting surface P of the housing 1 being in contact with or adjacent to the skin of human.

Next, an operational sequence of the laser depilatory device 100 will be described. First, a user connects to the power receiving connector 15 of the housing 1 the power supply connector 18 to which the power supply code 19 is connected. Subsequently, the power switch 14 is turned on. With the power switch 14 being turned on, the controller 17 supplies to the laser light generating section 11 the power supplied through the power supply code 19, the power supply connector 18, the power receiving connector 15 and the AC/DC converting section 16. At this moment, the controller 17 cutouts the power supply from the battery 13 to the laser light generating section 11.

When the power switch 14 is turned on and the power receiving connector 15 of the housing 1 is not connected to the power supply connector 18 to which the power supply code 19 is connected, the controller 17 supplies the direct-current power from the battery 13 to the laser light generating section 11.

Subsequently, in order to start the removal operation of the skin hair, the user directs the recessed portion 4 of the housing 1 towards the position on the skin on which the skin hair is to be removed. Further, the user abuts or approximates the contacting surface P of the housing 1 against or to his/her skin. The detection section 12 detects that the contacting surface P he in contact with or adjacent to the skin. Based on the detection thereof, the controller 17 allows the laser light generating section 11 to he driven. Consequently, the laser light is emitted from the respective laser light outlets 2 towards the respective heating elements 3 which oppose the laser light outlets 2, respectively.

The housing 1 is moved along the surface of the skin in a sliding manner so that the skin hairs grown on the skin are guided with the slits 6 and 7 to pass through inside the recessed portion 4. At this moment, the slit 6 or slit 7 allows the skin hairs passing through therein to be raised or groomed. Then the skin hairs are guided into the recessed portion 4 by the slit 6 or the slit 7 and then to be cut to an even length during the later hair removal operation.

### (Cauterization of Skin Hair)

The laser light cauterizes the skin hairs which have passed through the slit 6 or the slit 7 and been guided into the recessed portion 4 at a prescribed height.

When the skin hair is cauterized, the heating element 3 irradiated with the laser light is heated by the laser light emitted from the laser light outlet 2. The skin is moderately warmed that is adjacent to the skin in contact with the heated heating element 3. As a result, the pore is made open, and the hair removal can be achieved from closer position with respect to the skin. Additionally, the heating element 3 functions as a light absorbing element. In other words, the laser light is not reflected in other direction from the heating element 3. Accordingly, it can be prevented that the laser light enters into an eye of the user so that the safety can be ensured.

After the skin hair is removed by the above described operations, the contacting surface P is disengaged from the skin by operating the housing 1. As a result, the detecting section 2 detects that the housing 1 be distant from the skin. Based on the detection by the detecting section 2, the controller 17 allows the laser light generating section 11 to halt its driving. This enables the cauterization of the Skin hair with the laser light to halt.

At this moment, when the user turns off the power switch 14, the power supply can be halted from the battery 13 or the AC/DC converting section 16 to the laser light generating section 11. Finally, when the power supply connector 18 is pulled out from the power receiving connector 15, a sequence of the hair removal operation can be terminated. When the user does not turn off the power switch 14 and gets the contacting surface P to be in contact with or approximate the skin once again, the hair removal operation can be resumed or continued.

It should be noted that a part of the skin hairs removed by the cauterization is once accumulated within the recessed portion 4. During or after the hair removal operation, the housing 1 is to be operated such that the recessed portion 4 is directed downwardly. By doing so, the accumulated skin hairs can be easily eliminated from the recessed portion 4.

As described above, with the provision of slits 6 and 7 in the laser depilatory device 100, the slits 6 and 7 serve as a guide to smoothly guide the skin hair into the recessed portion 4 when the housing 1 is moved along the skin. At this moment, the slit 6 or slit 7 allows the skin hair to he raised or groomed. Then, since the laser light is emitted in parallel with the direction of travel of the housing 1, the skin hair that has passed through the slit 6 or slit 7 can he cauterized such that the skin hair is aligned at the prescribed height over the length in the prescribed direction.

Within the recessed portion 4, a part of the skin hair is irradiated with the laser light to be intensively cauterized so that each of skin hairs irradiated with the laser light has a cauterized surface having homogeneous and of uniformed shape. The laser light outlets 2 are linearly arranged (arranged in a straight line) at an equal interval along the rim portion of the recessed portion 4 so that wide range of skin hairs can be irradiated with the laser light. As a result, rapid hair removal treatment can he achieved.

In the laser depilatory device 100, the heating element 3 absorbs the laser light emitted from the laser light outlet 2 and then is heated. The heating element 3 generates heat by converting the light energy to the heat energy. Heat generation by the heat element 3 allows a pore of the skin contacting the heat element 3 to open. As a result, the hair removal elect can be improved.

### Modification to First Embodiment

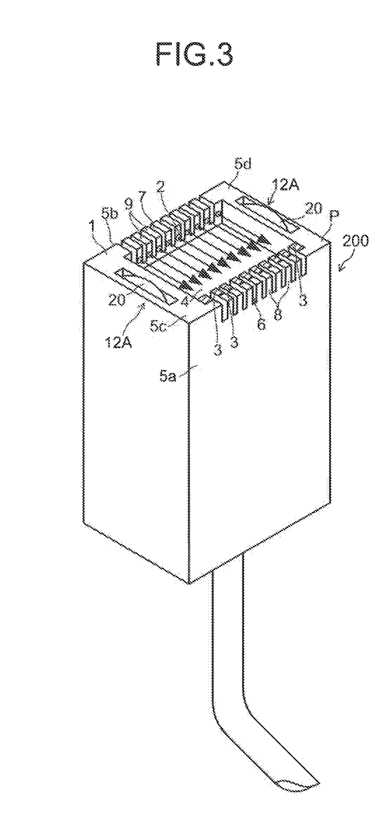

Hereinafter, referring to FIG. 3, the laser depilation device 200, the modification to the first embodiment, will be described. In this modification, a roller type detecting section 12A is employed in place of the detecting section 12 shown in FIG. 1. The roller type detecting section 12A detects that a roller 20 has rotated when the housing 1 is operated manually and the contacting surface P is moved in contact with or adjacent to the skin. The roller type detecting section 12A outputs to the controller 17 a signal indicating that the roller 20 has rotated (hereinafter referred to as “rotation detecting information”). In response to the reception of the rotation detecting information, the controller 17 controls the turn on/off of driving the laser light generating section 11. Since the turn on/off of an driving of the laser light generating section 11 is controlled based on the detection of the rotation detection information, the laser light is not to be emitted unless the housing 1 is in contact with or adjacent to the skin and then moved. This prevents the laser light from being emitted unnecessarily. As a result, the power consumption required for generating the laser light can be suppressed.

Preferably, the roller type detecting section 12A may output the rotation detecting information to the controller 17 only when the roller 20 has rotated in the specified direction. For example, the roller type detecting section 12A outputs the rotation detecting information to the controller 17 only when the housing 1 is moved towards the direction of emission of the laser light emitted from the laser light outlet 2 (the direction indicated with the arrow in FIG. 3).

In this manner, the roller type detecting section 12A outputs the rotating detection information to the controller 17 only when the roller 20 has rotated in one direction so that the laser light can be automatically emitted when, for example, the housing 1 is moved forward in the direction of emission of the laser light on the skin. In contrast, the emission of the laser light can be automatically halted when the housing 1 is stopped from moving or is moved backward.

Without provision of the roller type detecting section 12A, when the housing 1 is moved hack and forth, the laser light is emitted in response to the back and forth movement of the housing 1. Accordingly, the laser light may be irradiated repetitively onto the same skin hair more than necessary. As a result, it may be occurred that length of remaining skin hairs are uneven after the cauterization with the laser light or the keratin of the skin is damaged. In contrast, with provision of the roller type detecting section 12A, it can be prevented that the laser light be irradiated repetitively onto the same skin hair more than necessary. Also, it can be prevented that the keratin of the skin he damaged. Furthermore, with provision of the roller type detecting section 12A, the power consumption with continuous emission of the laser light can be suppressed.

### Second Embodiment

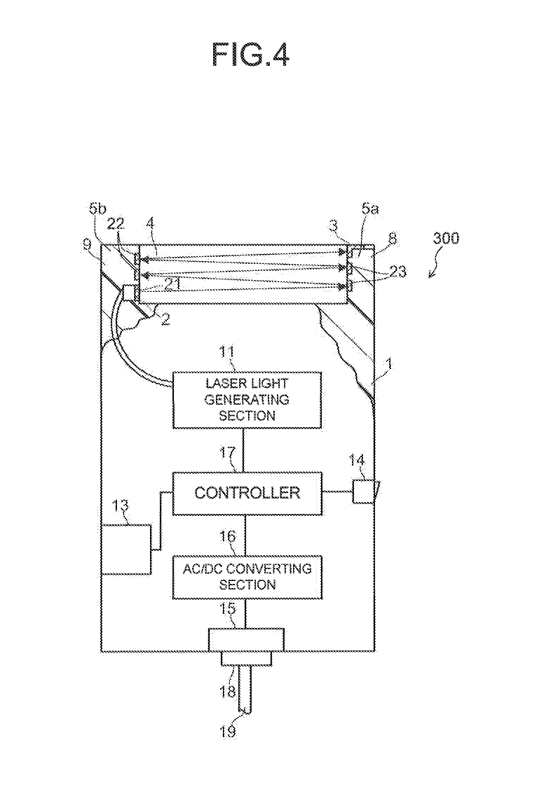

Hereinafter, referring to FIG. 4, the laser depilation device 300, the second embodiment, will be described. In the description hereinafter, the same element as the laser depilation device 100 according to the first embodiment is denoted by the same reference sign and duplicative description will be omitted.

The laser depilation device 300 according to the second embodiment is provided with a half mirror 21 at an aperture portion of the laser light outlet 2. Two mirrors 22 with the prescribed dimension and the prescribed shape are arranged at an equal interval at the wall section 5b in the direction of the aperture portion of the recessed portion 4 from the half mirror 21.

As such, two mirrors 23 are arranged at an equal interval at the wall section 5a in the direction of bottom portion of the recessed portion 4 from the heating element 3. The half mirror 21, the mirrors 22 and 23 are arranged such that the half mirror 21, the mirrors 22 and 23 are positioned in the optic axis of the laser light traveling forward with transmitting or reflecting thereamong.

With provision of the half mirror 21, it can be prevented that, when the laser light emitted from the laser light outlet 2 is reflected towards the laser light outlet 2 by the mirror 23, the reflected laser light is unintentionally entered into the laser light outlet 2.

The laser light emitted from the laser light outlet 2 first passes through the half mirror 21. Subsequently, the laser light is reflected by the mirror 23 at the lowermost part provided at the protruding portion 8. Yet subsequently, the laser light is reflected by the minor 22 at an upper part provided at the protruding portion 9. Yet subsequently, the laser light is reflected by the other mirror 23 provided at the protruding portion 8. Ultimately, the laser light is irradiated onto the heating element 3 provided at the protruding portion 8.

The skin hairs that are guided by either the slit 6 or the slit 7 to be introduced into the recessed portion 4 are cauterized by the laser light which is repetitively reflected by the minors 22 and 23. In this laser depilatory device 300, the skin hairs are cauterized at five points in the direction of the travel of the laser light on a light path. As a result, the hair removal can be achieved in more efficient manner. As described above, it is particularly efficient when the energy of the laser light generated by the laser light generations section 11 to employ a configuration where the laser light is reflected by, e.g., the mirrors 22 and 23.

As described above, the first and second embodiments and the modification to the first embodiment has been described. However, it is not limited to the above described embodiments. In other words, the various modifications can be applied to the embodiment. Hereinafter, the first to third modifications to the embodiment will be described.

### First Modification

For example, a laser diode (LD) can be directly disposed at the laser light outlet 2. The LD and the laser light generation section 11 are electrically connected and the laser light is irradiated onto the heating element 3. With employing this configuration, the light deriving tube 11A can be omitted. As a result, manufacturing cost of the laser depilatory device 100 or the like can be reduced.

### Second Modification

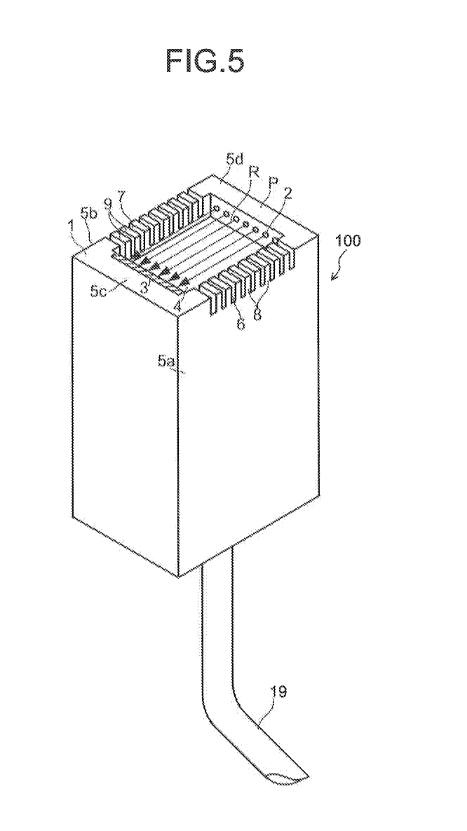

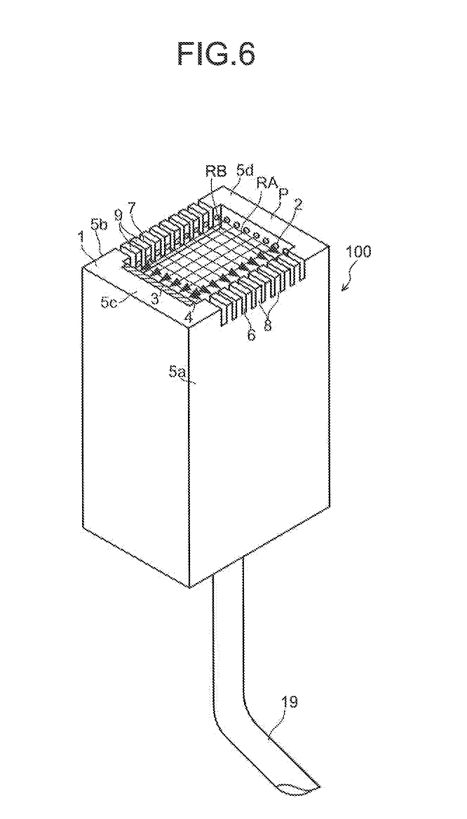

As shown in FIGS. 5 and 6, the layout position of the laser light outlet 2 can be modified appropriately. For example, the laser light outlet 2 can be disposed at the wall section 5d, instead of the wall section 5b as shown in FIG. 5. With employing this configuration, the laser light is irradiated onto the skin hair from the direction orthogonal to the above described direction of the travel of the housing 1 (i.e., the moving direction of the skin hair). It should be noted that in this case the heating element 3 is arranged at the wall section 5c. Needless to say, the laser light outlet 2 can be disposed at the wall section 5c. in this case, the heating element 3 is arranged at the wall section 5d.

As shown in FIG. 6, the laser light outlet 2 can be disposed at the wall sections 5b and 5d. In this case, the rays of laser light emit orthogonally each other. In other words, the hair removal treatment can be carried out by both the laser light RB which is emitted from the laser light outlet 2 disposed at the wall section 5b and the laser light RA which is emitted from the laser light outlet 2 disposed at the wall section 5d and orthogonal to the emitting direction of the laser light RB. Accordingly, the chance where the skin hairs are irradiated with the laser light is enhanced because the laser light outlet 2 is disposed not only at the wall section 5b but also further at the wall section 5d. Accordingly, the skin hairs can be cauterized in more efficient manner. Preferably, the layout positions of the laser light outlets 2 at the wall sections 5b and 5d are different each other in order to prevent the laser light RA and RB interfere (for example, the laser light outlet 2 at the wall section 5d is to be disposed at lower position of the recessed portion 4 in FIG. 6, in comparison with the laser light outlet 2 at the wall section 5b).

Yet another modification regarding the layout position of the laser light outlet 2 in FIG. 5 or 6. The laser light outlet 2, in addition to the wall section 5d, can be disposed at the wall section 5c opposing the wall section 5d. In this case, preferably, the laser light outlets 2 are disposed at the wall sections 5c and the 5d alternatively. Hereinafter, the term “disposed alternatively” means that the laser light outlet 2 is not disposed in the region of one wall section (for example, the wall section 5d) corresponding to the layout position of the laser light outlet 2 at the other wall section (for example, wall section 5c) in order to prevent the rays of laser light R emitted from the opposing laser light outlets 2 from conflicting each other. In other words, when the laser light outlet 2 is disposed at the wall section 5c from which the laser light is emitted from the wall section 5c towards the wall section 5d, the other laser light outlet 2 is not disposed in the region of the wall section 5d to which the laser light R emitted from the laser light outlet 2 reaches. Instead, the laser light outlet 2 from which the laser light R is emitted from the wall section 5d towards the wall section 5c is disposed in the region of the wall section 5d towards which the laser light R emitted from the laser light outlet 2 disposed at the wall section 5c does not reach. Needless to say, the configuration of the laser light outlets 2 which are alternatively disposed can be applied to the wall sections 5a and 5b.

### Third and Fourth Modifications

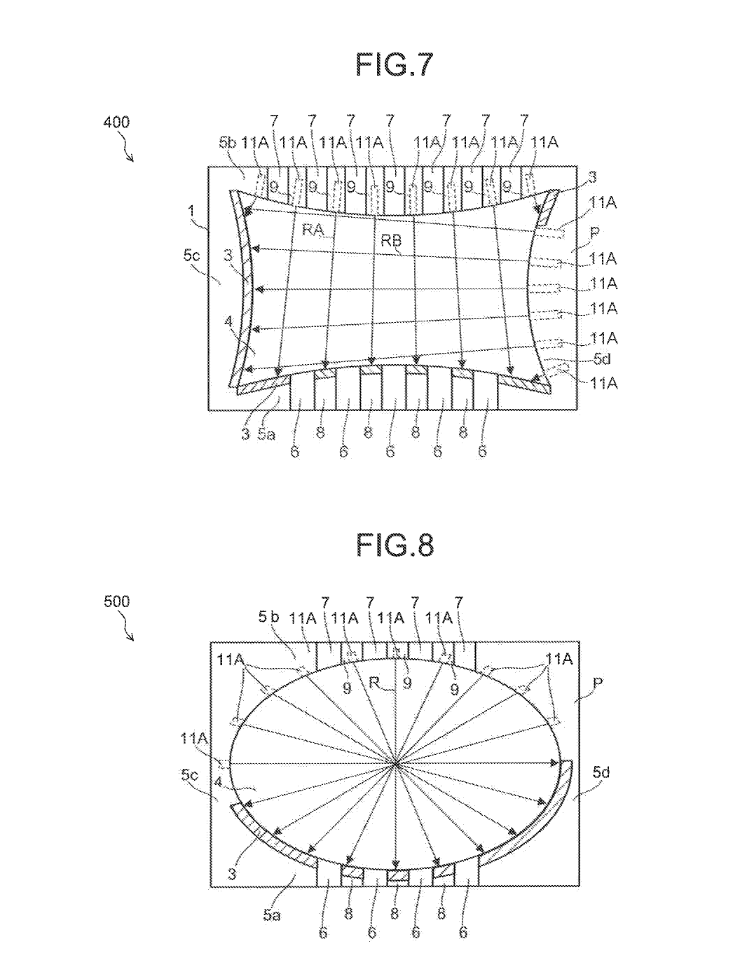

The shape of the wall sections 5a. to 5d and the emitting direction of the laser light can be modified appropriately, as the laser depilatory devices 400 and 500 shown in the FIGS. 7 and 8.

Hereinafter, referring to FIGS. 7 and 8, the third and fourth modifications where the shape of the wall sections 5a to 5d and the emitting direction of the laser light are modified will be described.

First, referring to FIG. 7, the laser depilatory device 400, which is the third modification, will be described. FIG. 7 is the view showing the laser depilatory device 400 viewed from straight above thereof. As shown in FIG. 7, in the laser depilatory device 400, a part of the wail sections 5a to 5d is formed in an arch-like shape. More particularly, the opposing surfaces of the wall sections 5a to 5d are formed such that the opposing surfaces are convex in an arch shape towards the direction of the corresponding wall sections 5a to 5d, respectively.

The light deriving tube 11A is disposed such that the end section thereof from which the laser light RA and RB are derived is along with the shape of the wall sections 5a to 5d. As a result, the rays of the laser light derived by the light deriving tube 11A (RA and RB in the Figs.) are emitted in the different direction each other towards the heating element 3 disposed at either one of opposing wall sections 5a to 5d. Accordingly, the rays of laser light RA and RB are emitted to spread in a fan-like shape as a whole, it should be noted that in the laser depilatory device 400, as described in the modification to the laser device 100 shown in the FIG. 6, preferably, the layout position of the laser light outlets 2 is different each other at the wall section 5b and the wall section 5d, in order to prevent the rays of laser light RA and RB to interfere each other.

According to the laser depilatory device 400, since a part of the wall sections 5a to 5d is formed in an arch-like shape, the area of the contacting surface P is increased. As a result, the area for contacting the skin surface is increased. Consequently, when the skin hair is to be removed, the stability of the laser depilatory device 400 with respect to the skin surface can be increased.

Next, referring to FIG. 8, the laser depilatory device 500, which is the fourth modification, will be described. FIG. 8 is a view Showing the laser depilatory device 500 viewed from straight above thereof As shown in FIG. 8, in the laser depilatory device 500, a part of the wall sections 5a to 5d is formed in an arch-like shape. More particularly, the opposing surfaces of the wall sections 5a to 5d are formed such that the opposing surfaces are concave in an arch-like shape in the direction of the corresponding wall sections 5a to 5d, respectively. As a result, the recessed portion 4 is formed in a substantially circular shape (in an elliptical shape in FIG. 8) as a whole.

The light deriving tube 11A is disposed such that the end section thereof from which the laser light R is derived is along with the shape of the wall sections 5a to 5d. The laser light R derived by the light deriving tube 11A is emitted towards the heating element 3 disposed at either one of opposing wail sections 5a to 5d. At this moment, the laser light R passes through the center part of the recessed portion 4.

In the laser depilatory device 500, as configured in this way, the derived laser light R passes through the center part of the recessed portion 4, as described above. Accordingly, the rays of laser light are synthesized in the center part of the recessed portion 4 at which the rays of laser light are concentrated to indicate a high energy state. Also, the interval between the rays of laser light becomes densely. As a result, an output of the laser light generated by the laser light generating section 11 can be set lower than the other embodiments. Consequently, the hair removal in the center part of the aperture can be carried out more strongly, while the safety is ensured.

What is claimed is:

1. A laser depilatory device comprising:
a housing including a contacting surface configured to contact with a skin surface and a recessed portion formed on the contacting surface;
a laser light generator disposed in the housing for generating laser light; and
a first outlet formed on an inner wall surface of the recessed portion, configured to emit the laser light through a space inside the recessed portion towards the inner wall.

2. The laser depilatory device according to claim 1, further comprising:
a second outlet formed on the inner wall surface of the recessed portion, configured to emit the laser light through the space inside the recessed portion towards the inner wall in a direction different from a direction of the laser light emitted from the first outlet,

3. The laser depilatory device according to claim 1,
wherein the first outlet is arranged in a straight line along the contacting surface.

4. The laser depilatory device according to claim 1, further comprising a slit opposing the first outlet.

5. The laser depilatory device according to claim 1, further comprising a heating element arranged substantially in parallel with the first outlet, the heating element being configured to partially contact with the skin surface, the heating element being configured to absorb the laser light emitted from the first outlet to generate heat.

6. The laser depilatory device according to claim 1, further comprising a tubular laser light deriving section connected to the laser light generator and the first outlet, configured to transmit the laser light generated by the laser light generator to the first outlet.

7. The laser depilatory device according to claim 1, further comprising:
a detector configured to detect a move of the contacting surface towards a direction of the laser light irradiated from the first outlet with respect to the skin surface; and
a controller configured to turn on/off an irradiation of the laser light based on a detection by the detecting section.
